(19) United States
(12) Patent Application Publication
(10) Pub. No.: US 2022/0246011 A1
(43) Pub. Date: Aug. 4, 2022

(54) METHODS, DEVICES, AND SYSTEMS FOR ROUND-THE-CLOCK HEALTH AND WELLBEING MONITORING OF INCARCERATED INDIVIDUALS AND/OR INDIVIDUALS UNDER TWENTY-FOUR-HOUR-SEVEN-DAY-A-WEEK (24/7) SUPERVISION

(71) Applicant: NC Seven Mountains, LLC, Clemmons, NC (US)

(72) Inventor: Bobby F. Kimbrough, Jr., Clemmons, NC (US)

(21) Appl. No.: 17/591,752

(22) Filed: Feb. 3, 2022

Related U.S. Application Data
(60) Provisional application No. 63/145,116, filed on Feb. 3, 2021.

(51) Int. Cl. G08B 21/02; G06F 1/16; G16H 40/67

(52) U.S. Cl. CPC G08B 21/02; G16H 40/67; G06F 1/163

(57) ABSTRACT

This disclosure presents methods, systems, and devices for monitoring individuals that are incarcerated and/or requiring twenty-four-hour-seven-day-a-week (24/7) supervision. According to one embodiment, a method is implemented on a wearable computing device. The method includes receiving (1) receiving a behavioral profile associated with an individual over a first wireless network, (2) receiving a medical profile associated with the individual over the first wireless network, (3) monitoring and storing first vital sign data of the individual, (4) detecting a first abnormality based on the first vital sign data; and (5) upon detecting the first abnormality, transmitting a first alert over a second wireless network.

### PRIORITY CLAIM

This application claims priority to U.S. Provisional Patent Application Ser. No. 63/145,116 filed Feb. 3, 2021, entitled “METHODS, SYSTEMS, AND DEVICEs FOR MONITORING THE HEALTH AND WELLBEING OF INDIVIDUALS THAT ARE INCARCERATED AND/OR UNDER TWENTY-FOUR-HOUR-SEVEN-DAY-A-WEEK (24/7) SUPERVISION,” (Attorney Docket No. 1238/2 PROV). The disclosure of the aforementioned application is incorporated herein by reference in its entirety.

### TECHNICAL FIELD

The present invention relates generally to wearable monitoring devices and systems. More specifically and disclosed herein are methods, devices, and systems for round-the-clock health and wellbeing monitoring of incarcerated individuals and/or individuals under twenty-four-hour-seven-day-a-week (24/7) supervision.

### BACKGROUND

The safety and wellbeing are of upmost importance for individuals that are incarcerated and/or under twenty-four-hour-seven-day-a-week (24/7) supervision. Suicide and attempted suicide are most difficult to detect and prevent. Even under the most stringent video and in-person monitoring situations current methods fail. As example, Jeffery Epstein on Aug. 10, 2018 committed suicide in the Metropolitan Correctional Center in New York City while awaiting trial. Jeffery Epstein was under 24/7 video surveillance and routine in-person checks. This high-profile case reinforced the difficulties in protecting an individual that intends to harm themselves.

Accidents (e.g., falls, etc.) and medical events (e.g., heart attacks, strokes, seizures, loss of consciousness, etc.) requiring quick response for individuals under 24/7 supervision are also of concern. Human monitoring techniques (including video monitoring) are too susceptible to human error and may have devastating outcomes for the individual when they cannot summon for help.

Inmates incarcerated in a county facility, a state facility, a federal facility, a private facility, or the like are at risk. Additionally, patients that are resident to psychiatric facility such as a long-term facility, a psych ward of a hospital, a respite facility, a memory-care facility, a group home, or the like are also at risk.

As such, new methods, systems, and devices are needed for monitoring the safety and wellbeing of individuals that are incarcerated and/or requiring 24/7 supervision.

### SUMMARY

This disclosure presents methods, systems, and devices for monitoring individuals that are incarcerated and/or requiring twenty-four-hour-seven-day-a-week (24/7) supervision. According to one embodiment, a method is implemented on a wearable computing device. The method includes receiving (1) receiving a behavioral profile associated with an individual over a first wireless network, (2) receiving a medical profile associated with the individual over the first wireless network, (3) monitoring and storing first vital sign data of the individual, (4) detecting a first abnormality based on the first vital sign data; and (5) upon detecting the first abnormality, transmitting a first alert over a second wireless network.

In some embodiments, the behavioral profile may include a first parameter associated with a risk of suicide and a second parameter associated with a risk of criminal incitement. In further embodiments, the medical profile may include a third parameter associated with a heart rhythm abnormality and fourth parameter associated a body type. In still further embodiments, the body type may be associated with a height-to-weight ratio. The body type may also be associated with an ectomorph body type, a mesomorph body type, and an endomorph type.

In some embodiments, the first vital sign data may include a blood oxygen level received from a pulse oximeter implemented within the wearable computing device. In other embodiments, the first vital sign data may include a pulse rate received from a pulse sensor implemented within the wearable computing device. In still other embodiments, the first vital sign data may include body temperature data received from a skin temperature sensor implemented within the wearable computing device.

In some embodiments, the method may include receiving a first alert trigger algorithm and detecting the first abnormality may be further based on the first alert trigger algorithm. The method may further include modifying the first alert trigger algorithm based on a machine learning algorithm. The first vital sign data may include a blood oxygen level received from a pulse oximeter implemented within the wearable computing device and the first alert trigger algorithm may include at least one weighting factor associated with a skin color of the individual.

According to another embodiment, a wearable computing device includes a processor, a memory, a first vital sign sensor, a first network interface, and a second network interface. The first wireless interface electrically is coupled with the processor and configured for wirelessly coupling to a first wireless network. The second wireless interface is electrically coupled with the processor and configured for wirelessly coupling to a second wireless network. The first vital sign sensor is electrically coupled with the processor. The memory is electrically is coupled with the processor. The memory includes program instructions configured for (1) receiving a behavioral profile associated with an individual over the first wireless network; (2) receiving a medical profile associated with the individual over the first wireless network; (3) monitoring and storing first vital sign data of the individual, (4) detecting a first abnormality based on the first vital sign data; and (5) upon detecting the first abnormality, transmitting a first alert over the second wireless network.

According to another embodiment, a non-transitory computer-readable storage medium stores instructions to be implemented on a wearable computing device including at least one processor. The instructions when executed by the at least one processor cause the wearable computing device to provide a method. The method includes receiving (1) receiving a behavioral profile associated with an individual over a first wireless network; (2) receiving a medical profile associated with the individual over the first wireless network, (3) monitoring and storing first vital sign data of the individual; (4) detecting a first abnormality based on the first vital sign data; and (5) upon detecting the first abnormality, transmitting a first alert over a second wireless network.

According to another embodiment, a method is implemented on a mobile device for provisioning a wearable computing device. The method includes (1) receiving a first unique identifier associated with the wearable computing device; (2) receiving a second unique identifier associated with an individual to be monitored using the wearable computing device; (3) transmitting a request to a server, over a first wireless network, for a behavioral profile and a medical profile associated with the individual using the second unique identifier; (4) transmitting a request to a server, over a first wireless network, for a behavioral profile and a medical profile associated with the individual using the second unique identifier; (5) receiving the behavioral profile and the medical profile over the first wireless network; (6) transmitting the behavioral profile and the medical profile to the wearable computing device over a second wireless network; and (7) receiving an acknowledgment the wearable computing device is operating normally and monitoring the individual using the behavioral profile and the medical profile.

According to another embodiment, a mobile device for provisioning a wearable computing device is disclosed. The mobile device includes a processor, a first wireless interface electrically coupled with the processor and configured for wirelessly coupling to a first wireless network, a second wireless interface electrically coupled with the processor and configured for wirelessly coupling to a second wireless network, and a memory electrically coupled with the processor. The memory includes program instructions configured for a method. The method includes (1) receiving a first unique identifier associated with the wearable computing device; (2) receiving a second unique identifier associated with an individual to be monitored using the wearable computing device; (3) transmitting a request to a server, over the first wireless network, for a behavioral profile and a medical profile associated with the individual using the second unique identifier; (4) receiving the behavioral profile and the medical profile over the first wireless network; (5) transmitting the behavioral profile and the medical profile to the wearable computing device over the second wireless network; and (6) receiving an acknowledgment the wearable computing device is operating normally and monitoring the individual using the behavioral profile and the medical profile.

According to another embodiment, a non-transitory computer-readable storage medium stores instructions to be implemented on a mobile device including at least one processor, a first wireless interface electrically coupled with the processor and configured for wirelessly coupling to a first wireless network, a second wireless interface electrically coupled with the processor and configured for wirelessly coupling to a second wireless network, and a memory electrically coupled with the processor. The instructions when executed by the at least one processor cause the mobile to provide a method for provisioning a wearable computing device. The method includes (1) receiving a first unique identifier associated with the wearable computing device; (2) receiving a second unique identifier associated with an individual to be monitored using the wearable computing device; (3) transmitting a request to a server, over the first wireless network, for a behavioral profile and a medical profile associated with the individual using the second unique identifier; (4) receiving the behavioral profile and the medical profile over the first wireless network; (5) transmitting the behavioral profile and the medical profile to the wearable computing device over the second wireless network; and (6) receiving an acknowledgment the wearable computing device is operating normally and monitoring the individual using the behavioral profile and the medical profile.

The features and advantages described in this summary and the following detailed description are not all-inclusive. Many additional features and advantages will be apparent to one of ordinary skill in the art in view of the drawings, specification, and claims presented herein.

### BRIEF DESCRIPTION OF THE DRAWINGS

The present embodiments are illustrated by way of example and are not intended to be limited by the figures of the accompanying drawings. In the drawings:

FIG. 1 depicts a diagram illustrating a bracelet (i.e., a wearable computing device) in accordance with embodiments of the present disclosure.

FIG. 2 depicts a block diagram of the bracelet of FIG. 1 in accordance with embodiments of the present disclosure.

FIG. 3 depicts a diagram illustrating a faceplate of the bracelet of FIG. 1 configured for monitoring an inmate in accordance with embodiments of the present disclosure.

FIG. 4 depicts a system diagram illustrating a plurality of bracelets being monitored for alerts by one or more central servers hosting one or more inmate monitoring server applications, a mobile device, and a workstation in accordance with embodiments of the present disclosure.

FIG. 5 depicts a mobile app flow chart executing on a mobile device for onboarding an inmate to a bracelet in accordance with embodiments of the present disclosure.

FIG. 6 depicts a flow chart for monitoring an inmate via a bracelet in accordance with embodiments of the present disclosure.

FIG. 7 depicts a block diagram illustrating the mobile device of FIG. 4 in accordance with embodiments of the present disclosure.

FIG. 8 depicts a block diagram illustrating the central server of FIG. 4 in accordance with embodiments of the present disclosure.

FIG. 9 depicts a block diagram illustrating the workstation of FIG. 4 in accordance with embodiments of the present disclosure.

### DETAILED DESCRIPTION

The following description and drawings are illustrative and are not to be construed as limiting. Numerous specific details are described to provide a thorough understanding of the disclosure. However, in certain instances, well-known or conventional details are not described in order to avoid obscuring the description.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

Unless otherwise indicated, all numbers expressing quantities of components, conditions, and so forth used in the specification and claims are to be understood as being modified in all instances by the term “about.” Accordingly, unless indicated to the contrary, the numerical parameters set forth in the instant specification and attached claims are approximations that can vary depending upon the desired properties sought to be obtained by the presently disclosed subject matter.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

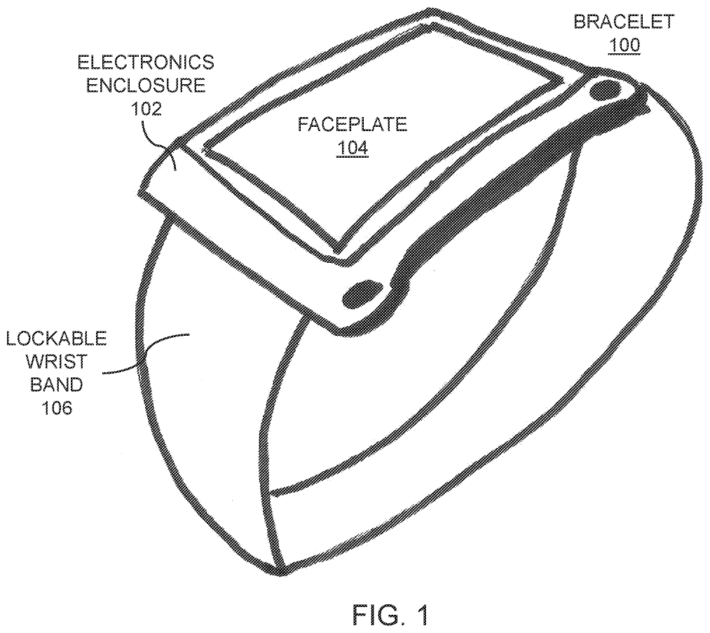

This present disclosure presents methods, systems, and devices for monitoring the safety and wellbeing of individuals that are incarcerated and/or requiring twenty-four-hour-seven-day-a-week (24/7) supervision. FIG. 1 depicts a diagram illustrating a bracelet 100 (i.e., a wearable computing device) in accordance with embodiments of the present disclosure. The bracelet 100 includes an electronic enclosure 102 with a faceplate 104. The bracelet 100 also includes a lockable wrist band 106. In other embodiments the lockable wrist band 106 may be replaced with a lockable ankle band (not shown in FIG. 1). The bracelet 100 is suitable for monitoring an inmate incarcerated in a county facility, a state facility, a federal facility, a private facility, or the like. The bracelet 100 is also suitable for monitoring a patient that is resident to psychiatric facility such as a long-term facility, a psych ward of a hospital, a respite facility, a memory-care facility, a group home, or the like. The bracelet 100 may also be applicable to home based monitoring, school based monitoring, or the like.

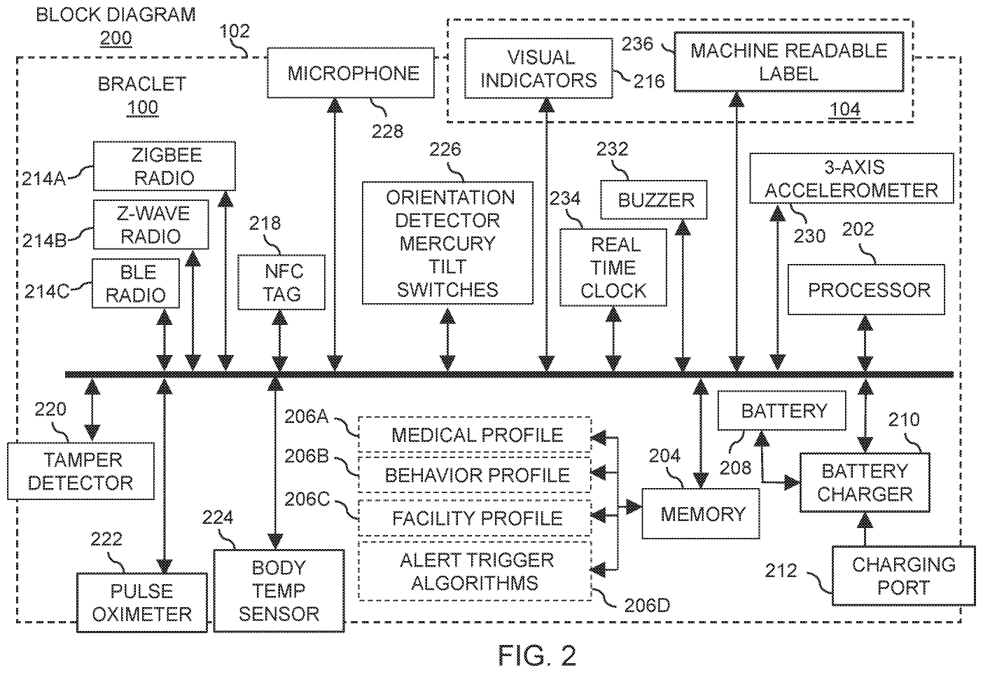

FIG. 2 depicts a block diagram 200 of the bracelet 100 of FIG. 1. The bracelet 100 includes a processor 202 and a memory 204 in accordance with embodiments of the present disclosure. In some embodiments, the memory 204 or a portion of the memory 204 may be integrated with the processor 202. The memory 204 may include a combination of volatile memory and non-volatile memory. In some embodiments the processor 202 and the memory 204 may embedded in a microcontroller. The processor 202 may be the Snapdragon® 4100 processor, the NXP Kinetix® microcontroller unit (MCU), or the like. The memory 202 may be configured to receive a medical profile 206A and a behavior profile 206B of an individual to me monitored for safety and wellbeing. The memory may also be configured to receive a facility profile 206C and one or more alert trigger algorithms 206D.

The bracelet 100 also includes a battery 208, a battery charger 210, and a charging port 212. The charging port 212 may be a wireless charging port. A plurality of radios for interfacing to multiple wireless networks is also implemented in the bracelet 100. The plurality of radios may include a Zigbee® radio 214A, a Z-wave® radio 214B, a Bluetooth® Low Energy (BLE) radio 214C, or the like. Visual indicators 216 are included on the faceplate 104 of the enclosure 102 and under control of the processor 202. The bracelet 100 includes a near field communication (NFC) tag 218 for secure identification to one or more NFC readers. In some embodiments, the bracelet 100 may include an NFC reader (not shown in FIG. 2) under control of the processor 202.

The bracelet 100 includes a tamper detector 220 configured for detecting if the individual being monitored attempts to remove, damage, or block communications, or block other monitoring capabilities of the bracelet 100. The bracelet 100 includes a pulse oximeter 222 and a body temperature sensor 224 for monitoring multiple vital signs of the individual including pulse rate, blood oxygen levels, and skin temperatures. The bracelet 100 also includes an orientation detector 226 including mercury tilt switches, a microphone 228, and a three-axis accelerometer 230. The orientation detector 226 is configured to detect a relative position to gravity of the bracelet 100. The three axis accelerometer 230 is configured to detect instantaneous movements on x, y, and z-axis of the bracelet 100. The microphone 228 may be used to detect voice communication from the individual and/or ambient sounds.

The bracelet 100 includes a buzzer 232 and/or vibration capability under control of the processor 202 and configured to gain the attention of the individual being monitored. The bracelet also includes a real time clock 234 for timestamping any monitored data and possibly changing alert trigger algorithms based on time-of-day and/or day-of-week. A machine readable label 104 is also implemented within and/or on the faceplate 104 of the enclosure 102. The machine readable label 236 may include a barcode, a quick response (QR) code, a radio-frequency identification (RFID) tag, or the like. In some embodiments, the RFID tag may be a smart RFID under control of the processor 202. The machine readable label 236 may be used by a smart phone, smart tablet, or the like for provisioning the bracelet 100 when on-boarding or off-boarding an individual under monitoring. In some embodiments, the machine readable label 104 may be replaced by using a BLE advertisement packet and/or the NFC tag 218 may be used for provisioning the bracelet 100.

In some embodiments, the bracelet 100 may include a global positioning system (GPS) receiver or the like (not shown in FIG. 2) readable by the processor 202 to identify a location of the bracelet 100. Also (not shown in FIG. 2), a plurality of analog-to-digital converters (ADCs) and digital-to-analog converters (DACs) may be used to interface the various components/sensors to the processor 202. In certain embodiments, the bracelet 100 may also include a speaker and associated amplifier circuitry (not shown in FIG. 2) for communicating directly to an individual being monitored. Additionally, the speaker may be tested remotely by sending a tone to the bracelet 100 via the ZigBee Radio 214A and/or the Z-wave radio 214B. The bracelet 100 may determine the tone is audible via the microphone 228 and returning a response via the ZigBee Radio 214A and/or the Z-wave radio 214B. When the speaker is not present in the bracelet 100, the individual may be communicated to via a public address (PA) system of the facility, while the individual's response would be monitored by the microphone 228 and returned over the ZigBee Radio 214A and/or the Z-wave radio 214B. The buzzer 232 may also be activated while the individual being monitored is being addressed by the facility PA.

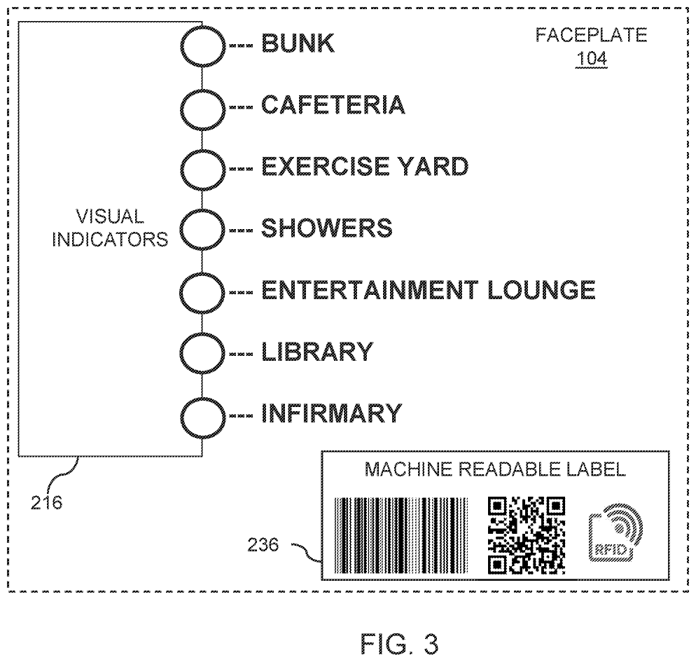

FIG. 3 depicts a diagram illustrating the faceplate 104 configured for monitoring an inmate in accordance with embodiments of the present disclosure. The visual indicators 216 may be used to communicate to an inmate an instruction to be at a current location and/or a next location (e.g., bunk, cafeteria, exercise yard, showers, entertainment lounge, library, infirmary, or the like). The visual indicators 216 may be light emitting diodes (LEDs). The visual indicators may be multiple colors for individual who cannot read (e.g., mental patients). The diagram also depicts the machine readable label 104 including a barcode, a QR code, and an RFID tag. The faceplate 104 may be configured to prevent intentional damage by the inmate being monitored and/or trip an alert associated with the tamper detector 220.

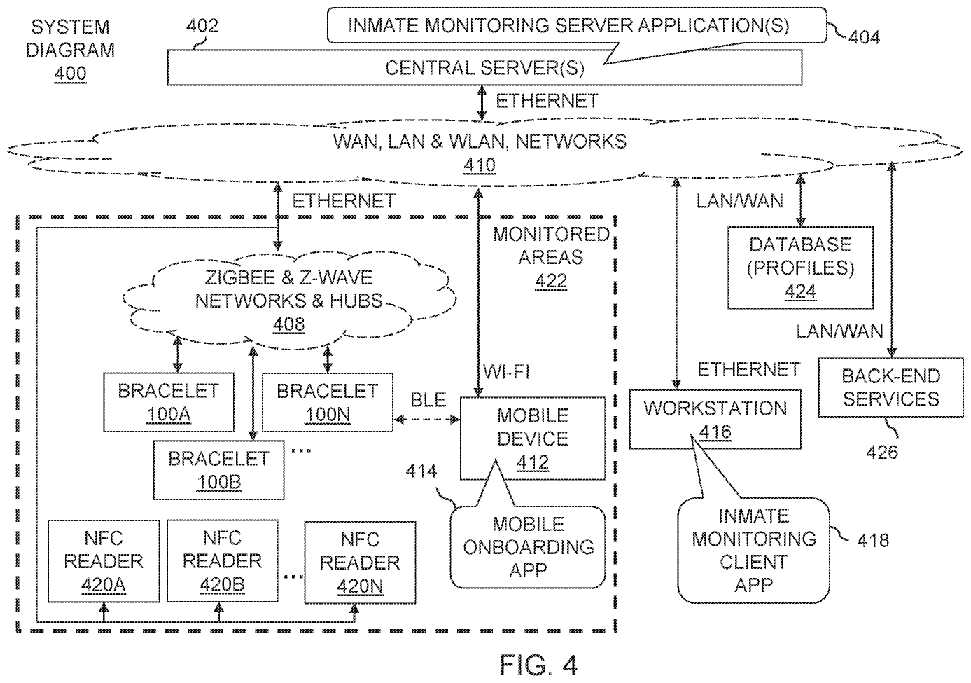

FIG. 4 depicts a system diagram 400 illustrating a plurality of bracelets 100A through 100N being monitored for alerts by one or more central servers 402 hosting one or more inmate monitoring server applications 404 in accordance with embodiments of the present disclosure. The plurality of bracelets 100A through 100N communicate with the one or more central servers 402 using Zigbee and Z-wave networks and hubs 408. The ZigBee® and Z-wave® networks and hubs 408 are coupled with the one or more central servers 402 using wide area networks (WANs) such as 2G, 3G, 4G, and/or 5G technologies networks including the Internet; local area networks (LANs) such as Ethernet; and/or wireless LANs such as Wi-Fi. The ZigBee® and Z-wave® networks and hubs 408 may allow over 65,000 unique addresses (e.g., bracelets 100) to be monitored. As such, the ZigBee® and Z-wave® networks and hubs 408 are much more scalable than the 256 maximum addresses of Wi-Fi and reach much further than Bluetooth® technologies within inmate facilities. ZigBee and Z-wave networks support mesh configurations and operate in different frequency spectrums (908 MHz for Z-wave and 2.4 GHz for ZigBee). As such, wireless connectivity and reliability between the bracelets 100A through 100N and the one or more central servers 402 are improved. Other wireless technologies such as 6LoWPAN (IPv6 over Low-Power Wireless Personal Area Networks), DigiMesh, or the like may be used for wireless connectivity and networking to/from the bracelets 100A through 100N.

The one or more central servers 402 may be local to the facility incarcerating inmates, mental patients, or the like. In other embodiment, the one or more central servers 402 may be within a cloud computing environment. The cloud computing environment may be a portion of the Microsoft Azure® cloud computing environment, the Amazon Web Services® (AWS) cloud computing environment, or the like. The one or more central servers 402 may be implemented as virtual servers such as an Ubuntu® server. The one or more central servers 402 may also be configured to be hosted within virtual containers. For example, the virtual container may be the Docker® virtual containers or the like. In some embodiments, the virtual servers and/or virtual containers may be distributed over a plurality of hardware servers using hypervisor technology.

A mobile device 412 (e.g., iPhone®, Android® device, or the like) may be configured to on-board the plurality of bracelets 420A-420N using a BLE interface and a mobile onboarding app 414 via Wi-Fi with the inmate monitoring server applications 404. A workstation 416 and an inmate monitoring client application 418 may communicate (e.g., Ethernet) with the one or more central servers 402 for instantaneous status (i.e. near real-time) of the plurality of bracelets 100A through 100N to facility staff. Additionally, the one or more central servers 402 may send push notifications directly to other mobile devices of the facility staff (not shown in FIG. 4).

A plurality of NFC readers 420A through 420N may be positioned at various locations within the monitored areas 422 and coupled with the one or more central servers 402 via Ethernet or other networking technologies. The plurality of NFC readers 420A through 420N may be used to allow inmates or the like being monitored by the plurality of bracelets 100A through 100N to check-in and check-out of the various locations (e.g., their bunk/sleeping area, the cafeteria, the exercise yard, the showers, the entertainment lounge, the library, the infirmary, or the like).

A database 424 may be hosted locally or remotely and used for storing profiles/etc. (e.g., medical profiles, behavior profiles, facility profiles, alert trigger algorithms, or the like. The inmate monitoring server applications 404 may also communicate with one or more back-end services 426. The back-end services 426 may include communication with off-site quick response teams (e.g., police, fire, rescue, or the like). Additionally, the back-end services 426 may also include communication with medical facilities (i.e., doctors, therapists, and/or the like) having physical and/or mental health responsibility for the monitored inmates. Additionally, the back-end services 426 may communicate with health insurance providers, facility insurance providers, local/state/federal agencies that require oversite. The back-end services 426 may also communicate via messaging with designated family members on a routine basis to give an overview of an inmate's overall wellbeing.

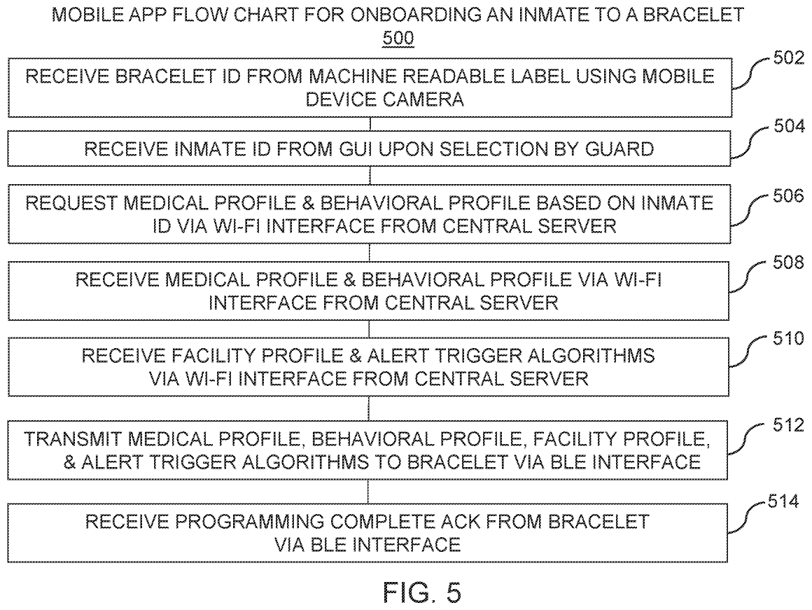

FIG. 5 depicts a mobile app flow chart 500 for onboarding an inmate to a bracelet 100 including steps 502 through 514 in accordance with embodiments of the present disclosure. For example, the mobile device 412 and mobile onboarding app 414 may be used.

In step 502, mobile device 412 receives a bracelet identifier (ID) that is unique to the bracelet 100. The bracelet ID may be received by a camera of the mobile device 412. In other embodiments the bracelet ID may be received by an RFID reader, an NFC reader, or the like embedded in the mobile device 312. In still other embodiments, the bracelet ID may be received as a BLE advertisement packet over a BLE interface of the mobile device 412.

In step 504, mobile device 412 receives an inmate ID from a graphical user interface (GUI) on the mobile device 412. The inmate ID may be selected from a pull-down menu or manually entered via the GUI.

In step 506, the mobile device 412 requests a medical profile 206A and a behavioral profile 206B using the inmate ID over a Wi-Fi connection. The medical profile 206A may be a generic profile or a unique profile associated with the given inmate. For example, the medical profile 206A may be a generic profile for a 60-70 year old male having a normal range body mass index (BMI). The behavior profile 206B may also be a generic profile or a unique profile associated with the given inmate. For example, the behavior profile 206B may be a generic profile for an inmate having sociopath antisocial personality disorder (ASPD) and/or other mental health condition.

In step 508, the mobile device 412 receives the medical profile 206A and the behavior profile 206B over the Wi-Fi connection from the one or more central servers 402 and/or the database 424.

In step 510, the mobile device 412 receives a facility profile 206C and alert trigger algorithms 206D over the Wi-Fi connection from the one or more central servers 402 and/or the database 424.

In step 512, the mobile device 412 transmits the medical profile 206A, the behavior profile 206B, the facility profile 206C, and the alert trigger algorithms 206D to the bracket 100 over the BLE interface.

In step 514, the mobile device 412 receives a programing complete acknowledgment from the bracelet 100. Additionally, the mobile device 412 may receive an indication the bracelet 100 has been correctly locked on the inmate and the tamper detector 220 is functioning correctly. The mobile device 412 may also receive initial indications from the pulse oximeter 222 and the body temp sensor that vital signs are being monitored correctly (i.e., the bracelet has been correctly installed on the inmate).

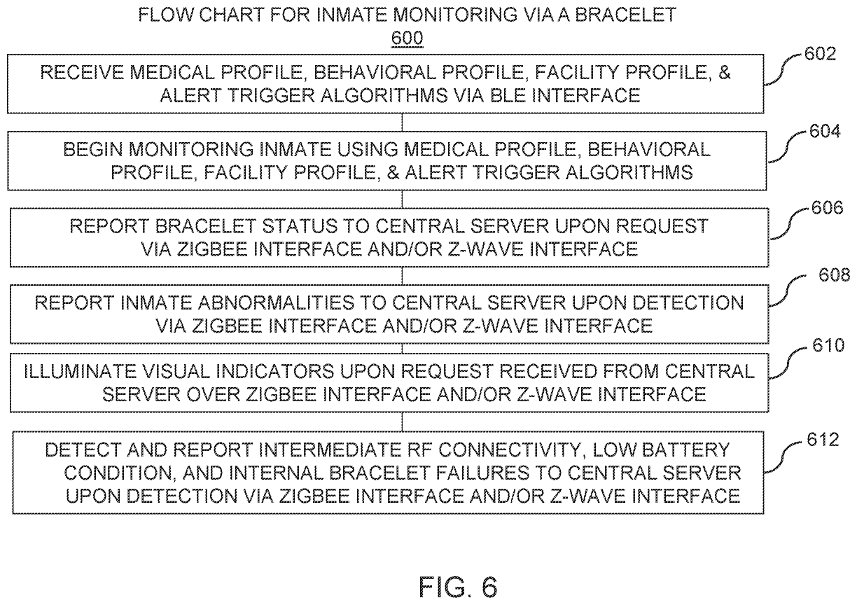

FIG. 6 depicts a flow chart 600 for monitoring an inmate via a bracelet 100 including steps 602 through 612 in accordance with embodiments of the present disclosure.

In step 602, the bracelet 100 receives the medical profile 206A, the behavior profile 206B, the facility profile 206C, and the alert trigger algorithms 206D to the bracket 100 over the BLE interface via a BLE interface from a mobile device 412 as disclosed in FIG. 5. The bracelet 100 may also send a confirmation to the mobile device 412 once the information is correctly loaded and preliminary vital sign information.

In step 604, the bracelet 100 begins monitoring the inmate and recording vital sign data including pulse rate data, blood oxygen level data, and skin temperature data. The vital sign data is continuously processed for abnormalities using the medical profile 206A, the behavior profile 206B, the facility profile 206C, and the alert trigger algorithms 206D. In some embodiments the one or more of the alert trigger algorithms 206D may be updated using one or more machine learning algorithms.

In step 606, the bracelet 100 routinely transmits a status to the one or more central servers 402 over the Zigbee and Z-wave networks and hubs 408. The status may include summary information from the pulse rate data, the blood oxygen level data, and the skin temperature data.

In step 608, the bracelet 100, upon detecting one or more abnormalities, transmits an abnormality alert to the one or more central servers 402 over the Zigbee and Z-wave networks and hubs 408. The abnormality alert includes the bracelet ID, a timestamp, and an alert type (e.g., low blood oxygen level, etc.)

In step 610, the bracelet 100 may receive a request from the one or more central servers 402 over the Zigbee and Z-wave networks and hubs 408 to illuminate one or more of the visual indicators 216 (e.g., bunk, cafeteria, exercise yard, showers, entertainment lounge, library, infirmary, etc.)

In step 612, the bracelet 100 may detect and report additional alerts to the one or more central servers 402 over the Zigbee and Z-wave networks and hubs 408. The alerts may include poor radio frequency (RF) connectivity over either the Zigbee network interface 214A and/or the Z-wave network interface 214B. In some embodiments, the bracelet 100 may alternate status reposts between the Zigbee network interface 214A and/or the z-wave network interface 214B base in current RF performance and/or signal levels. Other alerts may include a low battery condition and/or an internal bracelet failure.

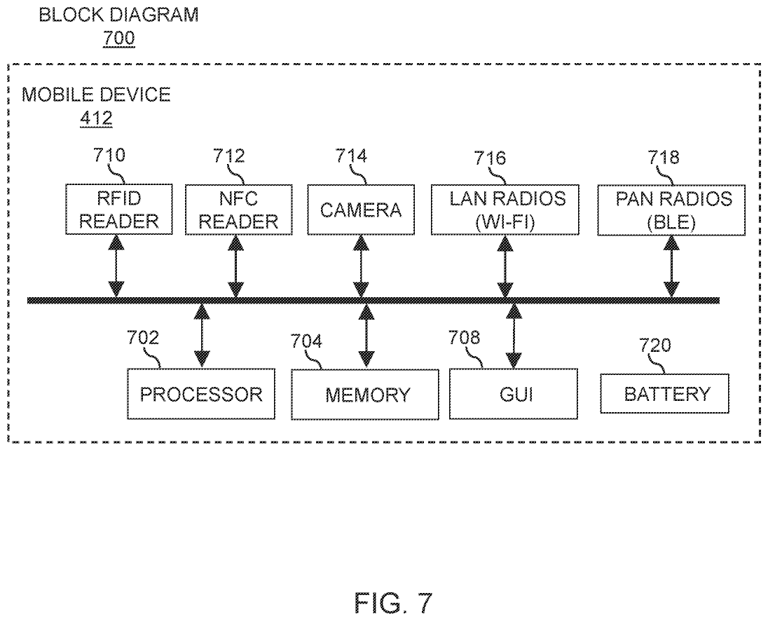

FIG. 7 depicts a block diagram 700 illustrating the mobile device 412 of FIG. 4 in accordance with embodiments of the present disclosure. The mobile device 412 may be a smart phone (e.g., cell phone), a tablet, a laptop, a smart watch, or the like. The mobile device includes a processor 702, a memory 704, a GUI 708, an RFIC reader 710, and NFC reader 712, a camera 714, LAN radios 716, PAN radios 718, and a battery 720.

In some embodiments the memory 704 or a portion of the memory 704 may be integrated with the processor 702. The memory 408 may include a combination of volatile memory (e.g., random access memory) and non-volatile memory (e.g., flash memory). The memory 704 includes program instructions (i.e., one or more mobile apps) when executed by the processor 702 allow the mobile device 412 to perform methods such as the flowchart 500 of FIG. 5. In certain embodiments, the processor 702 may be a mobile processor such as the Qualcomm® Snapdragon® mobile processor. For example, the processor 702 may be the Snapdragon® 855 mobile processor. The mobile device 402 also includes a user interface (UI) 410 and a display 412. The GUI 708 and may be a touchpad display.

The LAN radios 716 may include Wi-Fi technologies such as 802.11a, 802.11b/g/n, and/or 802.11ac circuitry. The PAN radios 718 may include Bluetooth® and/or BLE technologies for communication with the bracelet 100 as previously discussed. The mobile device may also include WAN radios (not shown in FIG. 7). The WAN radios may include 2G, 3G, 4G, and/or 5G technologies. The mobile device 412 may also include a GPS receiver (not shown in FIG. 7) for identifying and/or verifying its location.

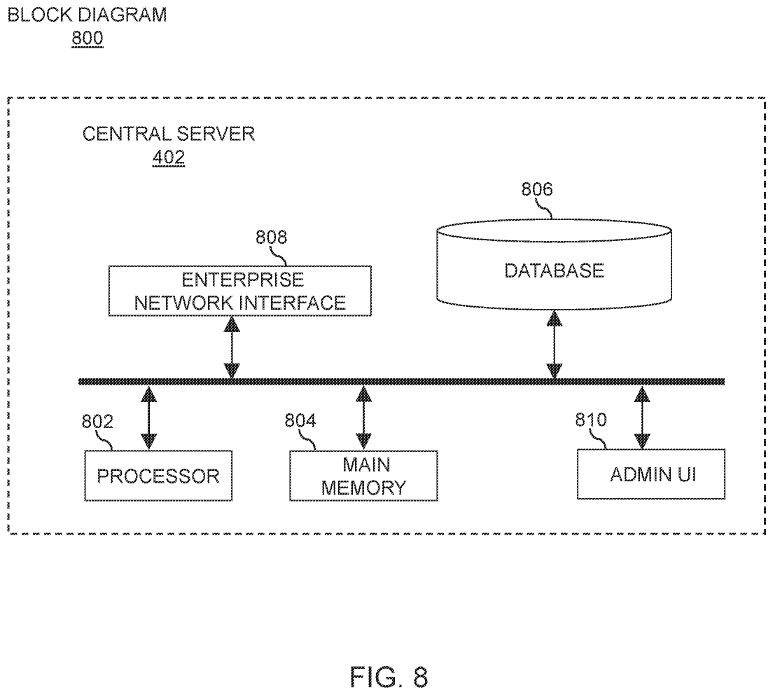

FIG. 8 depicts a block diagram 800 illustrating the central server 402 of FIG. 2 in accordance with embodiments of the present disclosure. The central server 402 may include at least one of processor 802, a main memory 804, a storage memory (e.g., database) 806, a datacenter network interface 808, and an administration user interface (UI) 810. The central server 402 may be configured to host an Ubuntu® server. In some embodiments Ubuntu® server may be distributed over a plurality of hardware servers using hypervisor technology.

The processor 802 may be a multi-core server class processor suitable for hardware virtualization. The processor may support at least a 64-bit architecture and a single instruction multiple data (SIMD) instruction set. The main memory 804 may include a combination of volatile memory (e.g., random access memory) and non-volatile memory (e.g., flash memory). The database 806 may include one or more hard drives.

The datacenter network interface 808 may provide one or more high-speed communication ports to the data center switches, routers, and/or network storage appliances. The datacenter network interface 808 may include high-speed optical Ethernet, InfiniBand (IB), Internet Small Computer System Interface (iSCSI), and/or Fibre Channel interfaces. The administration UI may support local and/or remote configuration of the central server 402 by a datacenter administrator.

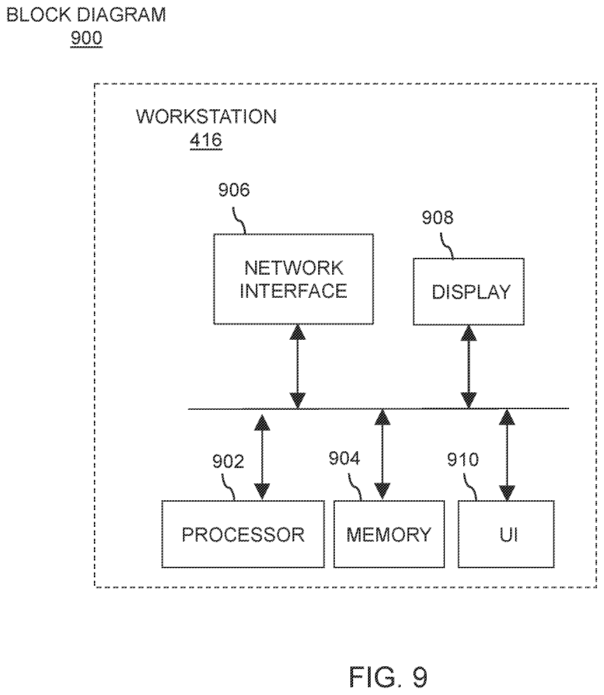

FIG. 9 depicts a block diagram 900 illustrating the workstation 416 of FIG. 4 in accordance with embodiments of the present disclosure. The workstation 416 may include at least one processor 902, a memory 904, a network interface 906, a display 908, and a UI 910. The memory 904 may be partially integrated with the processor 902. The UI 910 may include a keyboard and a mouse.

In certain embodiments the previously described methods, devices, and systems may provide detection for one or more of the following:
An individual attempting suicide (e.g., data indicating low oxygen level from the pulse oximeter).
An individual having a medical emergency (e.g., a seizure, a heart attack, a stroke, a seizure, etc.)
An individual committing a physical and/or verbal assault on another individual (e.g., data collected from orientation detector 226 and the three-axis accelerometer 230).
An individual that is currently being physically and/or verbally assaulted by another individual or a group of individuals (i.e., a rapid rise in pulse rate).

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including object oriented and/or procedural programming languages. Programming languages may include, but are not limited to: Ruby, JavaScript, Java, Python, Ruby, PHP, C, C++, C#, Objective-C, Go, Scala, Swift, Kotlin, OCaml, or the like. The program code may execute entirely on the user's computer, partly on the user's computer, as a stand-alone software package, partly on the user's computer, and partly on a remote computer or entirely on the remote computer or server.

Aspects of the present invention are described in the instant specification with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions.

These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. Thus, for example, reference to “a user” can include a plurality of such users, and so forth. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method implemented on a wearable computing device; the method comprising:
receiving a behavioral profile associated with an individual over a first wireless network;
receiving a medical profile associated with the individual over the first wireless network;
monitoring and storing first vital sign data of the individual;
detecting a first abnormality based on the first vital sign data; and
upon detecting the first abnormality, transmitting a first alert over a second wireless network.

2. The method of claim 1, wherein the behavioral profile includes a first parameter associated with a risk of suicide and a second parameter associated with a risk of criminal incitement.

3. The method of claim 2, wherein medical profile includes a third parameter associated with a heart rhythm abnormality and fourth parameter associated a body type.

4. The method of claim 3, wherein the body type is associated with a height-to-weight ratio.

5. The method of claim 1, wherein the first vital sign data includes a blood oxygen level received from a pulse oximeter implemented within the wearable computing device.

6. The method of claim 1, wherein the first vital sign data includes a pulse rate received from a pulse sensor implemented within the wearable computing device.

7. The method of claim 1, wherein the first vital sign data is a body temperature received from a skin temperature sensor implemented within the wearable computing device.

8. The method of claim 1 further comprising receiving a first alert trigger algorithm and detecting the first abnormality is further based on the first alert trigger algorithm.

9. The method of claim 8 further comprising modifying the first alert trigger algorithm based on a machine learning algorithm.

10. The method of claim 9, wherein the first vital sign data includes a blood oxygen level received from a pulse oximeter implemented within the wearable computing device and the first alert trigger algorithm includes at least one weighting factor associated with a skin color of the individual.

11. The method of claim 1 further comprising receiving a facility profile and detecting the first abnormality is further based on the facility profile.

12. The method of claim 1 further comprising receiving motion data from an accelerometer embedded in the wearable computing device and detecting the first abnormality is further based on the motion data.

13. The method of claim 12 further comprising receiving position data from an orientation detector embedded in the wearable computing device and detecting the first abnormality is further based on the position data.

14. The method of claim 12 further comprising receiving audio data from a microphone embedded in the wearable computing device and detecting the first abnormality is further based on the audio data.

15. The method of claim 14, wherein the microphone is configured to detect ambient background noise.

16. The method of claim 12 further comprising a tamper alert from a tamper detector embedded in the wearable computing device and detecting the first abnormality is further based on the tamper alert.

17. The method of claim 1, wherein the first wireless network is a personal area network compliant to at least one version of the Bluetooth® communication standard.

18. The method of claim 1, the second wireless network is compliant to at least one version of the ZigBee® communication standard.

19. A wearable computing device comprising:
a processor;
a first wireless interface electrically coupled with the processor and configured for wirelessly coupling to a first wireless network;
a second wireless interface electrically coupled with the processor and configured for wirelessly coupling to a second wireless network;
a first vital sign sensor electrically coupled with the processor; and
a memory electrically coupled with the processor, wherein the memory includes program instructions configured for:
receiving a behavioral profile associated with an individual over the first wireless network;
receiving a medical profile associated with the individual over the first wireless network;
monitoring and storing first vital sign data of the individual using the first vital sign sensor;
detecting a first abnormality based on the first vital sign data, the behavioral profile and the medical profile; and
upon detecting the first abnormality, transmitting a first alert over a second wireless network.

20. A non-transitory computer-readable storage medium, the non-transitory computer-readable storage medium storing instructions to be implemented on a wearable computing device including at least one processor, the instructions when executed by the at least one processor cause the wearable computing device to perform a method for:
receiving a behavioral profile associated with an individual over a first wireless network;
receiving a medical profile associated with the individual over the first wireless network;
monitoring and storing first vital sign data of the individual;
detecting a first abnormality based on the first vital sign data, the behavioral profile, and the medical profile; and
upon detecting the first abnormality, transmitting a first alert over a second wireless network.
